(19) United States
(12) Patent Application Publication
(10) Pub. No.: US 2006/0193928 A1
(43) Pub. Date: Aug. 31, 2006

(54) Novel herbal compositions and process for preparation thereof

(76) Inventors: Girish Sudhakar Soman, (US); Shrikrishna Govind Phadke, (US); Autumn Blum, Gulfport, FL (US)

Correspondence Address: Arnold S. Weintraub;The Weintraub Group, P.L.C., Suite 240, 32000 Northwestern Highway, Farminton Hills, MI, 48334, US

(21) Appl. No.: 11/352,134

(22) Filed: Feb. 10, 2006

(30) Foreign Application Priority Data
Feb. 10, 2005 (IN) 142/MUM/2005

(51) Int. Cl. A61K 36/00

(52) U.S. Cl. 424/725

(57) ABSTRACT

A process for obtaining various useful extracts from at least one herb includes the steps of extracting lypophilic ingredients through a supercritical CO₂ extraction process to obtain a first extract; subjecting the residue thereof to a second supercritical CO₂ and alcohol solvent process to obtain slightly polar compounds which define a second extract and, any residue after the second extraction is subjected to water extraction to obtain a still further extract. The various three extracts are mixed together in various combinations to define further extracts and which are admixed with potentiating therapeutic powders to enhance the potentiated therapeutic powders. The therapeutic powders when admixed with one or more extracts may be encapsulated in a vegetarian gelcap.

### BACKGROUND OF THE INVENTION

### Field of the Invention

The present invention relates to potentiating a therapeutic powder of herb/herbs by adding “beneficial extractives either individually or in combination” containing all the temperature sensitive lypophylic active ingredients which are free from toxic solvent residues and slightly polar and water soluble extractives. The present invention further relates to novel compositions comprising the potentiated therapeutic powder of herb/herbs comprising beneficial extractives of herb/herbs and raw powder of herb/herbs thereby obviate the need of inactive carrier. The present invention further relates to a process of extracting beneficial extractives from the selected herbs preferably comprising all the temperature sensitive lypophylic active ingredients which are free from toxic solvent residues and slightly polar and water soluble extractives.

There are several known herbs in India, which are used traditionally in herbal formulations. The present way of using such herbs in various formulations can be broadly classified as under
i) Use of herbs freshly harvested to make decoctions or fresh juice and formulating the same in various combinations as defined in the Ayurvedic texts.
ii) Use of herbs or parts of herbs after harvesting/drying under shed and powdering such herbs or parts of herbs and mixing such herbs together in the form of powder and using the same for the health care applications (either as a preventive, curative or supportive to human or animal health as supplementary or therapeutic).
The administering the mixture of dried herb powders, in required dosages for getting the full benefit of the therapeutic properties of the herbs is very difficult due to high daily dosage requirement. Hence, several better method of making herbal formulations have tried to improve the dosage as described below.
iii) First such improvement is by making “Extracts” of such powders from concentrates of such herbs made in water and using all the extractive of such herbs for beneficial applications in herbal health care and herbal dietary supplements in various forms like powders, capsules, tablets etc. Here, it has been observed in many scientific evaluations, that in spite of improving the dosage of “water soluble” or only “Highly Polar” ingredients, the formulations did not offer remarkable benefit of the equivalent dosage of herbal powders or mixtures of the herbal powders. The probable reason is the herbs are consisting of several “Highly Polar” (water soluble), “Slightly polar” (soluble in alcohol) and “Non Polar” (lypophylic) constituents, which act either individually or collectively in a synergistic manner. How and why this happens is still largely unknown to the scientific community. So selective use of “Water Soluble” or only “Polar” ingredients do not give the comparable benefits of equivalent dosage of herb powders reported in the earlier evaluations.
iv) These shortcomings were improved upon by U.S. Pat. No. 5,494,668. As described therein, the polar solvents were used initially for extraction and then subjected the residue to extraction using several hydrocarbon or petroleum solvents such as Hexane, Methanol, Petether, Acetone etc. Further such extractives made, using polar solvent (Water) and hydrocarbon solvents (petroleum solvents, which are slightly polar and non polar in nature) were combined together to form the “beneficial extracts” for using them together and mixing such “beneficial” extracts from various herbs together and mixing it with inert pharmaceutical carriers like Dicalcium phosphate (DCP) to offer in convenient dosage and form for use in various health care applications in capsules or tablet forms.
This method of making a combination of “Polar” and “Non Polar” beneficial extractives has following known shortcomings.
a) Due to subjecting the herb(s) or part of herb(s) to water extraction (for getting the “polar” beneficial extracts), the herb powder is subjected to high temperatures. This destroys many heat sensitive elements in the process. In many cases, the composition of some natural molecules in the herbs changes dramatically due to hydrolysis. For example, when Clove bud is subjected to heat, the eugenol acetate is converted in eugenol or when Sandalwood powder is subjected to heat the santelyl acetate is converted into santalol because of hydrolysis. Many such minor heat sensitive ingredients are either completely destroyed or change the form thereby reducing the beneficial therapeutic effects of the herbs.
b) Another major shortcoming is use of hydrocarbon petroleum solvents for extracting the herb powders/residues of herb powders after removing the polar (water soluble) extractives from the herb. The extracts obtained using such solvents like methanol, hexane, petroleum ether, acetone, toluene etc. are found to contain residues of such solvents. The residues of solvent even at ppm levels, administered orally to animals/humans are potential carcinogens or toxins.
c) The extraction carried out using such solvents, also has one more serious shortcoming. The temperature at the time of separation of extractives from the solvents is always more than the extraction temperature. This means that the extractives are subjected to higher temperature at the time of separation. This destroys many temperature sensitive ingredients present in the extract. The solvents also some times react with the constituents and change the form of many ingredients.
d) The idea of using such extractives together along with pharmaceutical carriers like DCP (Di Calcium Phosphate) or Malto Dextrin means administration of non-therapeutically beneficial ingredients to the object (animal/human). This defeats the purpose of administering the maximum therapeutically beneficial ingredients to the object. Due to this excess load on the body/system of the object of “non beneficial”, “non-absorbable” matter having no pharmacological/biochemical activity, results in lowering the efficacy of the formulations.

These shortcomings are eliminated by supercritical fluid extraction method, which are reported in WO0072861, U.S. Pat. No. 6,761,913, U.S. Pat. No. 6,576,274, U.S. Pat. No. 6,352,728, US 200212710 and US 20020197341, etc.

WO072861 describes methods of extracting and purifying bioactive substances from various plants and herbs, such as Kava root, Byrsonima species, Aesculus californica, Crataegus mexicana, Simmondsia chinensis, Pfaffia species, Alternanthera repens, Bursera species, Turnera species, Perezia species, Heimia salicifolia, Psidium species, Enterlobium species, Ptychopetalum olacoides, Liriosma ovata, and Chaunochiton kappleri, using supercritical fluid extraction and/or fluorocarbon solvent extract. These extracts are further used in formulation.

U.S. Pat. No. 6,761,913 describes the compositions and methods for the prevention and treatment of pain, inflammation and gastrointestinal irritation. The compositions comprise a purified, biologically active extract of celery seed, and may further be co-formulated with an additional analgesic or anti-inflammatory compound. The biologically active extract of celery seed is prepared by supercritical fluid extraction. An alcoholic extract of fresh celery seed is mixed with a suitable adsorbant, and then subjected to supercritical fluid extraction. Optionally, the resulting product is further treated or fractionated.

U.S. 200212710 and US 20020197341 describe physiologically synergistic mixtures of pomegranate extracts along with pomegranate seed oil and methods of use thereof. The pomegranate extract was prepared by supercritical fluid extraction.

There is no prior art which reports method of potentiating the therapeutic herb/herbs powder by using beneficial extractives prepared by supercritical fluid extraction and further use in health care formulation.

### OBJECTIVE

The main objective of the present invention is to provide a sustainable, ecologically beneficial method of potentiating the therapeutic herb/herbs powder and using them in healthcare formulations, with “beneficial extractives either individually or in combinations” containing all the temperature sensitive lypophylic active ingredients which are free from toxic solvent residues and slightly polar and water soluble extractives.

### SUMMARY OF THE INVENTION

According to the present invention, there is provided a method for potentiation of powder of herb/herbs by adding “beneficial extractives either individually or in combination” containing all the temperature sensitive lypophylic active ingredients which are free from toxic solvent residues and slightly polar(alcohol soluble) and polar water soluble extractives and a method for using the potentiated therapeutic powder of herb/herbs, in health care applications.

A method of potentiating the therapeutic powder of herb/herbs by adding beneficial extractives either individually or in combination comprising cleaning and shed drying the selected raw herb(s) for a particular health benefit; pulverizing it into powder form; mixing the extracts C and W of the herb/herbs in different proportion described in the following examples to potentiate the selected raw herb/herbs powder made as described above along with Aerated silica to make a homogeneous highly potentiated herb powder(s) mixture in a free flowing homogeneous powder form; formulating the free flowing homogeneous potentiated herb powder/combination of powders in tablets, capsules (Gelatin) or capsules (Vegetarian), which is a convenient form for use in health care applications.

The present invention further relates to novel compositions comprising the potentiated therapeutic powder of herb/herbs comprising beneficial extractives either individually or in combinations.

According to the present invention, there is provided a process of extracting beneficial extractives from the selected herbs preferably comprising all the temperature sensitive lypophylic active ingredients which are free from toxic solvent residues and slightly polar and water soluble extractives.

According to the present invention, a process of extracting beneficial extractives from the selected herbs preferably comprising all the temperature sensitive lypophylic active ingredients comprises cleaning the herb/herbs and powdering the herb/herbs; subjecting the herb/herbs to supercritical CO2 extraction to obtain the extract containing all temperature sensitive lypophilic (non polar) ingredients (Extract A) soluble in supercritical CO2; subjecting the residual herb after the supercritical supercritical CO2 extraction to extraction using supercritical CO2 and ethyl alcohol as a co-solvent and obtaining an extract containing alcohol soluble (slightly polar) beneficial ingredients (Extract B); combining extract A and B to get Extract C; and subjecting the residual herb/herbs obtained after getting extract B to water extraction and obtaining the extract in powder form containing polar ingredients (Extract W).

### DETAILED DESCRIPTION

The present invention discloses a method of making potentiated therapeutic powder of herb/herbs by adding beneficial extractives from the selected herbs preferably comprising all the temperature sensitive lypophylic active ingredients which are free from toxic solvent residues and slightly polar alcohol soluble extractives and water soluble extractives.

The present invention discloses a method for potentiating the therapeutic powder of herb/herbs by adding beneficial extractives either individually or in combination containing all the temperature sensitive lypophylic active ingredients which are free from toxic solvent residues and slightly polar and water soluble extractives.

The present invention further discloses novel compositions comprising the potentiated therapeutic powder of herb/herbs comprising beneficial extractives either individually or in combinations.

The present invention discloses a process of extracting beneficial extractives from the selected herbs preferably comprising all the temperature sensitive lypophylic active ingredients which are free from toxic solvent residues and slightly polar and water soluble extractives.

The term “herb” used in the present invention here is intended to cover one or more than one herb depending upon the final health care application.

According to the present invention, a process for preparing novel herbal compositions comprises the following steps
I. A process of extracting beneficial extractives from the selected herb/herbs comprising:
   1. Cleaning the herb/herbs and powdering the herb/herbs: The herb or part of herb was dried under the shed after removing the external impurities. Such dried herb was powdered in particle size ranging between 0.001 to 4 mm. It was ensured that the powder contains moisture less than 12%. Such powdered herb was then subjected to Supercritical CO₂ extraction.
   2. Subjecting the herb/herbs to supercritical CO2 extraction at a pressure varying between 80 Bar (80 kg/cm²) and 300 Bar (300 kg/cm²) at a temperature ranging between 31° C. and 45° C. The CO₂ was passed through the herb for a period of 2-3 hours depending upon the size of the extractors and the quantity of herb loaded into the extractor at a time. The quantity of CO₂ to be pumped through the herb varies between 10 kg of CO₂/kg of herb to 40 kg of CO₂/kg of herb depending upon the solubility of lypophylic compounds present in the herb. The CO₂ carried the extractives to the separator where the pressure of CO₂ was reduced to pressure varying between 40 Bar to 65 Bar and temperature between 10° C. to 30° C. as required to separate the solute (extract) and the CO₂.
   This method of extraction known as Supercritical CO₂ extraction is the safest method of extraction for dried herbs. The extract thus obtained contains all the temperature sensitive minor ingredients present in the herb and also all the other lypophylic soluble compounds. This extract obtained is Extract A.
   3. Subjecting the residual herb powders after getting Extract ‘A’ to extraction using mixture of CO₂ and ethyl alcohol in proportion of 90 to 97% Supercritical CO₂ and 3 to 10% of ethyl alcohol. The extraction was carried out at the pressure ranging between 80 Bar and 300 Bar and temperature ranging between 31° C. to 45° C. The quantity of solvent pumped (CO₂+Ethanol) varies between 5 kg/kg of herbs to 40 kg/kg of herbs. The solute (extract) and ethanol were separated from the CO₂ on reducing the solvent pressure between 40 Bar and 65 Bar and temperature between 10° C. to 30° C. The ethyl alcohol laced with extract was collected from the separator. The mixture was then subjected to vacuum distillation for separating the ethyl alcohol completely from the solute (extract), which was Extract B.
   4. Combining the extract ‘A’ and extract ‘B’ to obtain Extract C. This combined extract is also termed as SCO2 extract, in the following examples.
   5. Subjecting the herb powder residue remainder from above extraction to water extraction to obtain the water-soluble extractives in a paste form. The extract obtained was dried in tray dryers/vacuum dryer or in spray drier to obtain free flowing powder extract. This is Extract W.

The above extraction method was applied on several herbs and the extracts C (Supercritical CO2 extracts combined with extracts obtained by Supercritical CO2 with Ethyl alcohol as co-solvent) was in liquid/viscous/paste form. The post supercritical water extract (Extract W) was also obtained from the same herbs. This was in a powder form.

According to the present invention, the process of extraction has following important benefits:
1. The combined extracts thus obtained contain all the temperature sensitive minor ingredients present in the herb/herbs, which can contribute to the therapeutic benefit of the herb/herbs.
2. The extracts do not contain any harmful solvent residues/carcinogens/toxins.
3. The extracts are more “wholistic” or “total” in terms of beneficial ingredients.

These solvent free extracts were combined together and used for potentiation of herbs which was used in health care applications.

Whole herb powders were used in therapeutic applications giving unique advantage of synergistic actions of many known/unknown ingredients in the herb, which may or may not be present in the selective “extracts” obtained from the herb. It is therefore very much beneficial to use “potentiated” herbal powder with such known “beneficial” extractives.

It is an accepted fact that many crops like Wheat, Rice, and Potatoes etc are successfully modified using Genetic modification (GM) technology for increasing the amount of “beneficial” nutrients in the produce. After several years of use, this approach, however, is being questioned as to whether the Genetic modification is safe or whether it will have long-term harmful effects.

According to the present invention, the method adopted for potentiating the herbs involves a physical process of extraction to get the “beneficial” extractives with a very similar organoleptic profile of the herbs without having any harmful solvent residues, which avoids need of genetic modification and use such extractives for potentiation of raw herb powders.

A method for potentiating therapeutic powder of herb/herbs by adding beneficial extractives either individually or in combination comprising selecting herb/herbs or part thereof to be used for its known health benefit and cleaning from extraneous impurities after drying the same under shed; powdering the herb; sieving the powder to get the raw herb powder of fineness between 0.01 to 1.5 mm and adding beneficial extractives either individually or in combination containing all the temperature sensitive lypophylic active ingredients which are free from toxic solvent residues and slightly polar and water soluble extractives to the raw herb powder.

Different such raw herb powders were used for different health benefits as described in the examples 1 to 15. This raw herb powder(s) were potentiated as described below.

The extractives (Extract C & Extract W) obtained from various herbs were mixed together and then mixed with specific herb/herbs powder of fineness ranging between 0.01 mm and 1.5 mm prepared as described above, along with aerated silica to make a free flowing powder.

A combination of at least two extractives of herbs (which are “most beneficial” extractives or herb in terms of pharmacological and biochemical activities) are mixed with at least one raw herb powder from the selected herbs for specific benefits to potentiate the therapeutica powder of herb.

Formulating the free flowing homogeneous potentiated powder of herb/herbs in tablets, capsules (gelatin) or capsules (Vegetarian), which is a convenient form for use in health care applications.

According to the present invention, the composition comprises a combination of at least two extractives of herbs (which are “most beneficial” extractives or herb in terms of pharmacological and biochemical activities) mixed with at least one raw herb powder from the selected herbs for specific benefits.

The main feature of the present invention is to administer “potentiated” herb powder having known benefit and “beneficial” extractives of the supporting herbs together to achieve better therapeutic effect.

Another important feature of the present invention is the use of raw powder of herb/herbs in the compositions thereby obviating the need of inactive carrier.

The raw herb powder (in which extracts were mixed together) was used instead of using any inert carrier like DCP or Malto Dextrin in the composition.

According to the present invention, the lypophylic non-polar extractives and slightly polar alcohol soluble extractives which are in liquid or paste form and polar extractives, which are in powder form, were combined together and were converted in to a uniform matrix.

According to the present invention, the raw herb powder used to improve therapeutic activity of the formulation, as it contain all the ingredients present in the herb, some of which may not be available to the body from the extracts which are likely to be contributing to the therapeutic benefits of the herbs.

According to the present invention, the use of such potentiated herb matrix eliminates the necessity of using inert carriers such as DCP/Malto Dextrin, which are useless in terms of therapeutic benefits. Our method will definitely offer a more beneficial effect of the herb powder(s).

According to the present invention, the extracts of different physical nature, (i.e. Extract C in liquid or paste form and Extract W in free flowing powder form) were combined and obtained a free flowing mixture; Aerated silica was used as a drying agent, which also is known to offer certain health benefits.

Advantages of the present invention
1. Retention of heat sensitive elements in extracts, for potentiating the herb(s) powder(s).
2. Elimination of harmful solvent residues from the pharmacologically beneficial extractives.
3. Elimination of “Non beneficial” inert carriers from the formulations.
4. Potentiation of herb powders without making any Genetic modification, so completely ecofriendly way of potentiation of herbs.
5. Aerated Silica used for combining the ingredients also offers health benefits making all the ingredients in the formulation “beneficial” for health care applications.

It will be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention. This can be used for health care applications for all animals as well as human.

The following examples are for the purpose of illustration of the invention only and are not intended in any way to limit the scope of the invention.

### EXAMPLE 1

Composition containing potentiated powder of Ashwgandha (Withania somnifera) and Shatavari (Asparagus racemosus) for energizing and relieving stress.

Ashwagandha root powder 25 to 100 mg and Shatavari root powder 25 to 80 mg potentiated with Ashwagandha root SCO₂ extract 5 to 25 mg (Extract C) and Ashwagandha root post supercritical hydro extract 80 to 200 mg (Extract W) with Shatavari root SCO₂ extract 1 to 10 mg (Extract C), and Shatavari root post-supercritical hydrophilic extract 100 to 480 mg (Extract W). In order to make this mixture free flowing, aerated silica 10 to 100 mg was thoroughly mixed together. This mixture was then used to make either tablets, Hard gelatin capsules or Veggie capsules 250 to 900 mgs each. This unique mixture of know adoptogens helps in enhancing energy levels while helping to alleviate fatigue. It promotes physical and mental health and helps to support the body's immune defenses and enhances longevity and helps building vitality when administered orally in desired dosages.

### EXAMPLE 2

Composition containing a mixture of potentiated powders of Basil Leaf (Ocimum sanctum) & Turmeric (Curcuma longa) cooked rhizome for supporting upper respiratory functions

Basil whole herb powder 55 to 190 mg and Turmeric rhizome powder 20 to 180 mg potentiated with Basil leaf CO2 extract 10 to 40 mg (Extract C), Basil leaf post supercritical extract 70 to 180 mg (Extract W), Turmeric rhizome SCO2 extract 35 to 90 mg (Extract C), Neem leaf (Azadirachta indica) SCO2 extract 2 to 17 mgs (Extract C), Long pepper (Piper longum) fruit SCO2 extract 2 to 22 mg (Extract C) and Long pepper post supercritical hydro extract 33 to 100 mgs (Extract W). In order to make this mixture free flowing, Aerated silica 10 to 100 mg was thoroughly mixed together. This mixture was then used to make either tablets, hard gelatin capsules or Veggie capsules 250 to 900 mg each. This mixture helps to elevate body defense against infections, to promote immunity and respiratory health, gives soothing effect to upper respiratory tract when administered orally in desired dosages.

### EXAMPLE 3

Composition containing potentiated powder of Turmeric (Curcuma longa) rhizome as anti-inflammatory agent and blood purifier.

Turmeric rhizome powder 150 to 600 mgs potentiated with Turmeric rhizome SCO2 extracts 40 to 110 mg (Extract C). In order to make this mixture free flowing, Aerated silica 30 to 100 mg was thoroughly mixed together. This mixture was then used to make either tablets, hard gelatin capsules or Veggie capsules 250 to 820 mgs each This mixture supports skin, health and luster, acts as blood purifier, helps to reduce inflammation, supports cardiovascular functions when administered orally in desired dosages.

### EXAMPLE 4

Composition containing potentiated powder of Tinospora cardifolia as immuno modulator & hepato protective agent.

Tinospora cardifolia stem powder 100 to 400 mg potentiated with Tinospora cardifolia root SCO2 extract 25 to 80 mg (Extract C), Tinospora cardifolia post supercritical hydro extract 90 to 330 mg (Extract W). In order to make this mixture free flowing, Aerated silica 10 to 100 mg was thoroughly mixed together. This mixture was then used to make either tablets, hard gelatin capsules or Veggie capsules 250 to 900 mg each. This mixture helps to regulate liver functions, improves immune system and body resistance against infections, promotes longevity when administered orally in desired dosages.

### EXAMPLE 5

Composition containing a mixture of Potentiated Brahmi (Bacopa monnieri) powder for imparting calm sleep and helping neuro functions.

Brahmi whole herb powder 100 to 280 mg potentiated with Brahmi leaves SCO2 extract 10 to 45 mg (Extract C), Brahmi leaves post supercritical hydrophilic extract 115 to 450 mg of (Extract W), Spikenard (Nardostachys jatamansi) SCO2 extract 2 to 30 mg (Extract C). In order to make this mixture free flowing, Aerated silica 20 to 100 mg is thoroughly mixed together. This mixture is then used to make either tablets, hard gelatin capsules or Veggie capsules 250 to 900 mg each. This unique mixture helps to reduce stress and anxiety, helps improve memory and focus, helps imparting restful calm sleep, and helps improve intelligence and other brain functions when administered orally in desired dosages.

### EXAMPLE 6

Composition containing a mixture of potentiated Pomegranate (Punica granatum) seed and Licorice (Glycyrrhiza glabra) powder for helping female patients suffering from Peri Menopausal syndromes.

Pomegranate seed powder 20 to 100 mg and Licorice powder 60 to 100 mg potentiated with Pomegranate seed SCO2 extract 20 to 65 mg (Extract C), Pomegranate post supercritical hydro extract 30 to 260 mg (Extract W), Licorice root SCO2 extract 4 to 20 mg (Extract C), Licorice root post supercritical hydrophilic extract 30 to 150 mg (Extract W), Ashwagandha root (Withania somnifera) SCO2 extract 3 to 28 mg (Extract C), Ashwagandha root post supercritical hydrophilic extract 70 to 170 mg (Extract W), Tribulus terestris root SCO2 extract 20 to 60 mg (Extract C) and Tribulus terestris root post supercritical hydro extract 30 to 90 mg (Extract W). In order to make this mixture free flowing, Aerated silica 20 to 100 mg was thoroughly mixed together. This mixture was then used to make either tablets, hard gelatin capsules or Veggie capsules 250 to 900 mg each. This unique mixture helps to minimize discomfort associated with PMS, helps to improve hormonal imbalance, helps to increase energy and vitality when administered orally in desired dosages.

### EXAMPLE 7

Composition containing potentiated Eclipta alba powder for migraine.

Eclipta alba powder 15 to 70 mg and Emblica powder 20 to 80 mg potentiated with Eclipta alba root SCO2 extract 7 to 33 mg (Extract C), Eclipta alba root post supercritical hydrophilic extract 100 to 280 mg (Extract W), Ginger (Zingiber officinale) rhizomes SCO2 extract 3 to 22 mg (Extract C), Emblica SCO2 extract 5 to 28 mg (Extract C) and Emblica hydrophilic extract 70 to 400 mg (Extract W). In order to make this mixture free flowing, Aerated silica 20 to 100 mg was thoroughly mixed together. This mixture was then used to make either tablets, hard gelatin capsules or Veggie capsules 250 to 900 mg each. This mixture of herb complex helps to support psychosomatic functions, support gastric and liver functions, reduce physical and mental stress, and neutralize the toxins accumulated in the body, thereby helping the patients suffering from migraine when administered orally in desired dosages.

### EXAMPLE 8

Composition containing potentiated Fenugreek (Trigonella foenum-graecum) powder for curing & supporting cardiovascular conditions.

Defatted Fenugreek seed powder 210 to 500 mg potentiated with Commiphora mukul SCO2 extract 25 to 100 mg (Extract C), Turmeric rhizome (Curcuma longa) SCO2 extract 40 to 120 mgs (Extract C), Tinospora cardifolia root SCO2 extract 5 to 35 mg (Extract C) and Tinospora cardifolia post-supercritical hydrophilic extract 65 to 115 mg (Extract W). In order to make this mixture free flowing, aerated silica 20 to 100 mg was thoroughly mixed together. This mixture was then used to make either tablets, hard gelatin capsules or Veggie capsules 200 to 900 mg each. This unique mixture helps to balance body metabolism to support healthy cholesterol levels and to support cardiovascular health when administered orally in desired dosages.

### EXAMPLE 9

Composition containing potentiated powder of Shatavari (Asparagus racemosus) for supporting womens health.

Shatavari (Asparagus racemosus) powder 50 to 200 mg potentiated with Shatavari root SCO2 extract 1 to 15 mg (Extract C), Shatavari root post supercritical hydrophilic extract 265 to 460 mg (Extract W), Hemidesmus indicus root SCO2 extract 23 to 70 mg (Extract C), Hemidesmus indicus post-supercritical hydro extract 75 to 170 mg (Extract W), Berberies aristata root SCO2 extract 7 to 25 mg (Extract C), Berberies aristata post supercritical hydro extract 16 to 47 mg (Extract W) and Long pepper (Piper longum) fruit SCO2 extract 1 to 10 mg (Extract C). In order to make this mixture free flowing, aerated silica 15 to 100 mg was thoroughly mixed together. This mixture was then used to make either tablets, hard gelatin capsules or Veggie capsules 200 to 900 mg each. This mixture helps to elevate vitality, energy and spirit, supports normal development and health of female reproductive system, this herbs complex will help to support pregnancy and lactation when administered orally in desired dosages.

### EXAMPLE 10

Composition containing mixture of potentiated Turmeric (Curcuma longa) & Ashwagandha (Withania somnifera) supporting cancer therapy & improving quality of life for cancer patients.

Turmeric powder 30 to 200 mg and Ashwagandha root powder 30 to 200 mg potentiated with Turmeric rhizome SCO2 extract 80 to 220 mg (Extract C), Ashwagandha root SCO2 extract 1 to 20 mg (Extract C), Ashwagandha root post supercritical hydro extract 80 to 230 mg of (Extract W), Tinospora cardifolia root SCO2 extract 5 to 45 mg (Extract C), Tinospora cardifolia post supercritical hydrophilic extract 50 to 260 mg (Extract W), Ginger (Zingiber officinale) rhizomes SCO2 extract 8 to 32 mgs (Extract C) and Neem leaf (Azadirachta indica) SCO2 extract 2 to 15 mgs (Extract C). In order to make this mixture free flowing, aerated silica 70 to 190 mg was thoroughly mixed together. This mixture was then used to make either tablets, hard gelatin capsules or Veggie capsules 500 to 900 mgs each. This unique mixture helps to support healthy and normal cell growth, prevent nausea and vomiting associated with cancer treatment. Turmeric and Ginger are known for COX-2 inhibition when administered orally in desired dosages.

### EXAMPLE 11

Composition containing potentiated Ashwagandha (Withania somnifera) & Tinospora cardifolia powder for treating Arthritis.

Ashwagandha (Withania somnifera) powder 50 to 200 mg and Tinospora cardifolia powder 50 to 200 mg, Boswellia extract powder 90 to 220 mgs, potentiated with Ashwagandha (Withania somnifera) root SCO2 extract 1 to 12 mg (Extract C), Ashwagandha root post-supercritical hydro extract 60 to 180 mg (Extract W), Tinospora cardifolia root SCO2 extract 10 to 38 mg (Extract C), Tinospora cardifolia 110 to 260 mg post-supercritical hydro extract (Extract W), Tribulus terestris root SCO2 extract 20 to 60 mg (Extract C), Tribulus terestris root 40 to 120 mg of post-supercritical hydrophilic extract (Extract W), and Ginger (Zingiber officinale) rhizomes SCO2 extract 3 to 22 mg (Extract C). In order to make this mixture free flowing, aerated silica 20 to 100 mg was thoroughly mixed together. This mixture was then used to make either tablets, hard gelatin capsules or Veggie capsules 200 to 900 mg each. This mixture helps to reduce inflammation, promotes healthy joints functions, used as mild analgesic containing herbal anti-aging phyto-nutrients that inactivate free radicals, promotes normal cell thereby improving the quality of life of patients suffering from arthritis when administered orally in desired dosages.

### EXAMPLE 12

Composition containing is mixture of potentiated Coriander (Coriandrum sativum) powder and Turmeric (Curcuma lona) powder to help allergic patients.

Turmeric rhizome powder 90 to 225 mg and Coriander seed powder 170 to 385 mg potentiated with Turmeric rhizomes SCO2 extract 40 to 115 mg (Extract C), Coriander seed SCO2 extract 50 to 135 mg (Extract C), Neem leaf (Azadirachta indica) SCO2 extract 3 to 20 mg (Extract C) and Black pepper (Piper nigrum) fruit SCO2 extract 3 to 22 mg (Extract C). In order to make this mixture free flowing, aerated silica 20 to 100 mg was thoroughly mixed together. This mixture was then used to make either tablets, hard gelatin capsules or Veggie capsules 200 to 900 mg each. This composition helps to improve energy at cellular levels, prevent infections and act as anti-allergy herbs when administered orally in desired dosages.

### EXAMPLE 13

Composition containing potentiated defatted Fenugreek (Trigonella foenum-graecum) powder and Turmeric (Curcuma longa) powder for supporting patients suffering from Type II diabetes

Defatted Fenugreek powder 250 to 400 mg and Turmeric powder 50-150 mg potentiated with Turmeric rhizomes SCO2 extract 50 to 120 mg (Extract C), Neem leaf (Azadirachta Incia) SCO2 extract 4 to 12 mg (Extract C), Tinospora cardifolia SCO2 extract 10 to 30 mg (Extract C), and Tinospora cardifolia post supercritical hydrophilic extract 70 to 180 mg (Extract W). In order to make this mixture free flowing, aerated silica 20 to 100 mg was thoroughly mixed together. This mixture was then used to make either tablets, hard gelatin capsules or Veggie capsules 200 to 900 mg each. This formulation helps to regulate the blood sugar levels, heart and liver functions and neuro functions, which are under constant stress in case of diabetic patients, when administered orally in desired dosages.

### EXAMPLE 14

Composition containing Turmeric (Curcuma longa) and Tinospora Cardifolia powders for heapto protection.

Turmeric powder 50 to 100 mg and Tinospora cardifolia powder 50 to 200 mg potentiated with Tinospora cardifolia SCO2 extract 4 to 25 mg (Extract C), Tinospora post supercritical hydrophilic extract 50 to 150 mg (Extract W), Turmeric (Curcuma longa) SCO2 extracts 25 to 75 mg (Extract C), Eclipta alba SCO2 extract 5 to 20 mg (Extract C), and Eclipta alba post supercritical hydrophilic extract 40 to 100 mg (Extract W). In order to make this mixture free flowing, aerated silica 20 to 100 mg was thoroughly mixed together. This mixture was then used to make either tablets, hard gelatin capsules or Veggie capsules 200 to 900 mg each. This formulation helps reduce discomfort to those suffering from hepatitis conditions when administered orally in desired dosages.

### EXAMPLE 15

Composition containing potentiated Mucuna pruriens & Ashwagandha (withania somnifera) powder for Men's health.

Ashwagandha powder 50 to 200 mg, Mucuna pruriens powder 50 to 200 mg and saffron powder 3 to 10 mg potentiated with Ashwagandha root SCO2 extract 5 to 20 mg (Extract C), Ashwagandha Post supercritical hydrophilic extract 50 to 150 mg (Extract w), Tribulus terestris SCO2 extract 10 to 50 mg (Extract C), Tribulus terestris post supercritical extract 25 to 75 mg (Extract W), Mucuna pruriens SCO2 extract 10 to 40 mg, and Talimkhana (Asterocantha longifolia) SCO2 extract 5 to 20 mg (Extract C). In order to make this mixture free flowing, aerated silica 20 to 100 mg was thoroughly mixed together. This mixture was then used to make either tablets, hard gelatin capsules or Veggie capsules 200 to 900 mg each. This helps to improve libido, increase strength and stamina and increases sperm count when administered orally in desired dosages.

### EXAMPLE 16

Composition containing potentiated Terminalia chebula powder for Weight management.

Terminalia chebula powder 50 to 150 mg potentiated with Terminalia chebula SCO2 extract 4 to 20 mg (Extract C), Terminalia chebula post supercritical hydrophilic extract 40 to 100 mg (Extract W), Terminalia bellerica SCO2 extract 2 to 10 mg (Extract C), Terminalia bellerica post supercritical hydrophilic extract 10 to 40 mg (Extract W), Vijaysar (asna) SCO2 extract 3 to 20 mg (Extract C), Pterocarpus Marsupium post supercritical hydrophilic extract 25 to 75 mg (Extract W), Ginger (Zingiber officinale) SCO2 extract 1 to 8 mg (Extract C), Black Pepper (Piper nigrum) SCO2 extract 1 to 8 mg (Extract C), Long pepper (Piper longum) SCO2 extract 1 to 8 mg (Extract C), Embellica ribes SCO2 extract 2 to 20 mg (Extract C), Cyperus rotundus SCO2 extract 2 to 20 mg (Extract C), Plumbago Zelanica SCO2 extract 2 to 15 mg (Extract C), Plumbago Zelanica post supercritical extract 5 to 25 mg. (Extract W). In order to make this mixture free flowing, aerated silica 20 to 100 mg was thoroughly mixed together. This mixture was then used to make either tablets, hard gelatin capsules or Veggie capsules 200 to 900 mg each. This helps to reduce absorption of fats, enhances fat metabolism, improves lipid profile and helps to reduce body weight when administered orally in desired dosages.

Having, thus, described the invention what is claimed is:

1. A process for extracting beneficial extractives from at least one selected therapeutic herb, comprising:
(a) cleaning the at least one herb;
(b) powdering the at least one herb;
(c) subjecting the at least one herb to supercritical CO₂ extraction to obtain a first solute and a first powder residue, the first solute defining a first extract;
(d) subjecting the first residue to extraction with a mixture of supercritical CO₂ and an alkanol to obtain a second solute and a second residue;
(e) removing the alkanol from the second solute to form a second extract, and
(f) subjecting the second residue to water extraction to obtain water soluble extractives, the extractives defining a third extract.

2. The process of claim 1 wherein the third extract is dried after extraction to form a free flowing powder.

3. The process of claim 1 which further comprises mixing together the first extract and the second extract to form a fourth extract.

4. The process of claim 1 wherein the first supercritical extraction extracts all temperature sensitive lypophilic components of the herb.

5. The process of claim 1 which further comprises mixing together the third and fourth extracts to form a fifth extract.

6. The process of claim 3 which further comprises:
mixing together the third and fourth extracts, the process further comprising mixing therewith aerated silica to form a free flowing powder.

7. The process of previous claim 6 which further comprises mixing together the first extract, the second extract and the fourth extract to form a uniform matrix.

8. The process of claim 1 wherein the herb is dried in a shed.

9. A method for potentiating a therapeutic powder of at least one herb which comprises:
admixing with the therapeutic powder a first, a second and a fourth extract,
encapsulating the admixture in a vegetarian capsule, and
wherein the extracts are obtained by the process of claim 3.

10. The method of claim 9 wherein the therapeutic powder is a raw herb powder.
